(19) United States
(12) Patent Application Publication
(10) Pub. No.: US 2025/0331707 A1
(43) Pub. Date: Oct. 30, 2025

(54) COMPOSITIONS AND METHODS FOR TREATING KIDNEY STONES

(71) Applicant: The Regents of the University of Michigan, Ann Arbor, MI (US)

(72) Inventors: Kristian Black, Ann Arbor, MI (US); Mahir Maruf, Ann Arbor, MI (US); Luke Fisher, Ann Arbor, MI (US); Annie Li, Ann Arbor, MI (US); Monique Lamoureux, Ann Arbor, MI (US); Tyler Tish, Ann Arbor, MI (US); Christopher Jeffers, Ann Arbor, MI (US)

(21) Appl. No.: 19/191,852

(22) Filed: Apr. 28, 2025

Related U.S. Application Data
(60) Provisional application No. 63/640,587, filed on Apr. 30, 2024.

(51) Int. Cl. A61B 1/018; A61B 1/307; A61B 17/00; A61B 17/22

(52) U.S. Cl. CPC A61B 1/018; A61B 1/307; A61B 17/22012; A61B 2017/0034; A61B 2017/00477; A61B 2017/00991

(57) ABSTRACT

Provided herein are devices, systems and methods for treating kidney stones. In particular, provided herein are devices for improving the safety and efficiency of percutaneous nephrolithotomy.

### CROSS-REFERENCE TO RELATED APPLICATION

This application claims the benefit of U.S. Provisional Application No. 63/640,587, filed Apr. 30, 2024, which is incorporated by reference herein in its entirety.

### FIELD

Provided herein are compositions and methods for treating kidney stones. In particular, provided herein are devices for improving the safety and efficiency of percutaneous nephrolithotomy.

### BACKGROUND

The primary percutaneous nephrolithotomy (PCNL) surgical tools to remove large kidney stones are difficult to manipulate and control and their improper usage can result in kidney damage, longer recovery times, and even additional surgeries. While this surgery is highly effective in completely removing a patient's large kidney stones, the main tools that are used to perform this surgery, a nephroscope and a lithotripter, can be difficult to use safely over extended periods of time. The use of these tools can lead to damage to the renal parenchyma that results in longer recovery periods and in major complication instances, additional surgery can be required.

Improved devices for performing PCNL are needed.

### SUMMARY

Provided herein are devices, systems, and methods for treating kidney stones. In particular, provided herein are devices for improving the safety and efficiency of percutaneous nephrolithotomy.

The devices and methods described herein improve the safety and efficiency of PCNL by providing improved control over nephroscopes and lithotripters during procedures.

For example, in some embodiments, provided herein is an assembly, comprising: a) a lithotripter attachment device comprising a lithotripter locking assembly; b) a nephroscope attachment device comprising a nephroscope locking assembly; and c) a telescoping connector connecting the lithotripter attachment device and the nephroscope attachment device.

In some embodiments, the lithotripter locking assembly comprises a first collar portion, a second collar portion, and a releasable latch; and wherein the latch holds the first collar portion and the second collar portion together when the latch is in a closed configuration. In some embodiments, the lithotripter attachment device further comprises one or more additional components selected, for example, a thumb groove, a locking pin, and a keyhole shaped opening. In some embodiments, the keyhole shaped opening is configured for insertion of a lithotripter into the keyhole shaped opening. In some embodiments, the lithotripter locking assembly secures a lithotripter in the assembly.

In some embodiments, the telescoping connector comprises a plurality (e.g., 1, 2, 3, 4, 5, or more) of sets of a plurality of outer telescope arms, a plurality of inner telescope arms and a plurality of biasing members (e.g., compression springs). In some embodiments, the telescoping connector further comprises one or more compression spring attachment holes. In some embodiments, the telescoping connector comprises a resting position where the outer telescope arms, inner telescope arms, and compression springs are fully extended and a closed configuration where the inner telescoping arms are inserted into the outer telescoping arms and the springs are compressed. In some embodiments, the telescoping connector moves between the resting and the closed configuration during use.

In some embodiments, the nephroscope attachment device further comprises one or more components selected, for example, a top handle and a bottom handle. In some embodiments, the nephroscope locking assembly comprises a set of a front plate, a back plate, tabs, magnets, and springs. In some embodiments, the front and back plates are held in place around a nephroscope by the magnets, wherein the nephroscope locking device includes a base with at least one handle, a first tab pivotably connected to the base about a first axis, a second tab pivotably connected to the base about a second axis. In some embodiments, the second axis is spaced apart from and parallel to the first axis. In some embodiments, the first tab includes at least one permanent magnet. In some embodiments, there is a bias member positioned between the base and the first tab.

Also provided is use of an assembly described herein to secure a lithotripter and a nephroscope (e.g., during percutaneous nephrolithotomy).

Further embodiments provide a method of performing percutaneous nephrolithotomy, comprising: a) securing a lithotripter and a nephroscope in an assembly described herein; and b) performing percutaneous nephrolithotomy with the lithotripter and a nephroscope. In some embodiments, the assembly provides control of the forward and backward movements of the lithotripter within the working channel of the nephroscope. In some embodiments, the assembly can be controlled with only hand and wrist movement.

Additional embodiments are described herein.

### BRIEF DESCRIPTION OF THE DRAWINGS

FIG. 1 shows a nephroscope and a lithotripter.

FIG. 2 shows a device of embodiments of the present disclosure attached to a nephroscope and lithotripter.

FIG. 3 shows components of a device of embodiments of the present disclosure.

FIG. 4 shows front, side, and back views of a device of embodiments of the present disclosure.

FIG. 5 shows an overview of a lithotripter attachment device of a device of embodiments of the present disclosure.

FIG. 6 shows details of the locking assembly of a lithotripter attachment device of a device of embodiments of the present disclosure.

FIG. 7 shows details and dimensions of the locking assembly of a lithotripter attachment device of a device of embodiments of the present disclosure.

FIG. 8 shows details and dimensions of the locking assembly of a lithotripter attachment device of a device of embodiments of the present disclosure.

FIG. 9 shows an overview of a telescope connector of a device of embodiments of the present disclosure.

FIG. 10 shows details of a telescope connector of a device of embodiments of the present disclosure.

FIG. 11 shows details of components of a device of embodiments of the present disclosure.

FIG. 12 shows details and dimensions of an arm of a telescope connector of a device of embodiments of the present disclosure.

FIG. 13 shows details of a nephroscope attachment device of a device of embodiments of the present disclosure.

FIG. 14 shows details of a nephroscope attachment device of a device of embodiments of the present disclosure.

FIG. 15 shows dimensions of a detailed view of a nephroscope attachment device of a device of embodiments of the present disclosure.

FIG. 16 shows dimensions of a detailed view of a nephroscope attachment device of a device of embodiments of the present disclosure.

### DETAILED DESCRIPTION

Provided herein are devices, system, and methods for treating kidney stones. In particular, provided herein are devices for improving the safety and efficiency of percutaneous nephrolithotomy.

PCNL first starts by making an incision in the patient's flank with the positioning of this incision varying based on where the stones are located in the patient's kidney. The nephroscope is inserted into the kidney until a view of the stone can be seen. For these surgeries, due to the compactness of the human kidney, the tip of the nephroscope is positioned within a centimeter of the stone that is being operated on. Once a clear view of the stone is achieved, the lithotripter is slowly inserted through the working channel of the nephroscope until the tip of it protrudes from the exit point of the working channel and can be seen on camera. The nephroscope and lithotripter together are then maneuvered to the stone and the lithotripter is carefully pushed further out of the exit point while the ultrasonic energy of the lithotripter is turned on using a foot pedal by the surgeon. When the tip of the lithotripter touches the stone the kidney stone is broken up into small fragments and suctioned up.

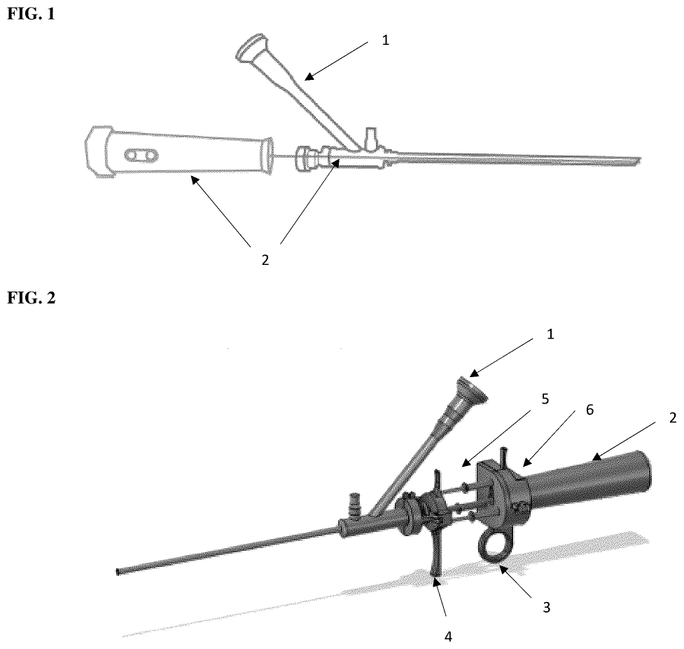

FIG. 1 shows a typical system for PCNL comprising a lithotripter and a nephroscope. One of the first issues with using these tools stems from the fact that the lithotripter needs to be inserted and removed frequently during a percutaneous nephrolithotomy as the surgeon switches between tools. Since the lithotripter probe is longer than the working channel of the nephroscope, the lithotripter cannot be fully inserted without puncturing the patient's kidney. Because of this, the device has to be inserted slowly and carefully, taking several seconds and noticeably decreasing the speed at which the surgery can be performed and therefore increasing the cost of surgery for the patient.

Secondly, due to how these devices interface with each other, it is difficult to precisely move the lithotripter forward and backward while it is protruding from the tip of the nephroscope. This is because the surgeon has one hand on the nephroscope and one hand on the lithotripter and moving your whole hand requires the movement of many parts of the arm such as the shoulder, elbow, and wrist. Due to engaging many parts of the arm, it makes it highly difficult to precisely move the lithotripter by the fractions of a millimeters that are required during these surgeries. Because of the difficulty of these precise movements, residents undergo extensive training while preparing to be a urology surgeon. Despite this extensive training, these fine motor movements are still difficult to execute and accidents can still occur. Additionally, engaging the whole arm is tiring for the surgeon.

Accordingly, provided herein are devices and methods for improving the safety and efficiency of PCNL. The devices of the present disclosure are designed to lock onto both the lithotripter and nephroscope to provide fine control of the forward and backward movements of the lithotripter within the working channel of the nephroscope. The device can be moved similar to a resectoscope, which uses only hand and wrist movements to control the surgery. This eliminates the fatigue of whole arm and shoulder movements, as well as increases precision needed for these surgeries. The added precision allows for millimeter scale movements, which is important to PCNL surgery, but currently very difficult with the normal tools. The device also allows for quick insertion and removal so that time can be saved during the surgery.

Exemplary assemblies and devices are shown in FIGS. 1-16.

In some non-limiting embodiments, all of the device's movement are controlled by the muscles within the hands and fingers as well as compression springs. In some embodiments, the proximal hold is a finger ring that the thumb will be placed in or a thumb groove. The thumb may also be positioned in the proximal ventral finger groove.

In some embodiments, the distal dorsal hold is for the index finger, and the distal ventral hold is for the middle and any number of the remaining fingers to be placed on (e.g., based on the surgeon's preference).

Forward motion is controlled by pressing with the thumb and the thumb groove and retraction of the lithotripter is provided by the pressure supplied by the compression springs placed between the lithotripter and nephroscope locking pieces. The locking mechanism for the lithotripter features an overhead clamp that then latches to the bottom piece to secure a tight fit. The nephroscope locking piece is held together by magnets to allow for quick insertion and removal of the device to the nephroscope which is important.

During initial usability testing the device passed tests including means for precise control, stability of lithotripter probe, secureness to the lithotripter and nephroscope, and others including ergonomic design and weight. The device allows for directly proportional movement between the surgeon's hand and the lithotripter with 0.7 mm of tolerance while maintaining stabilization of the same position with a 3 mm tolerance. This is much better than the current methods used for PCNL surgery.

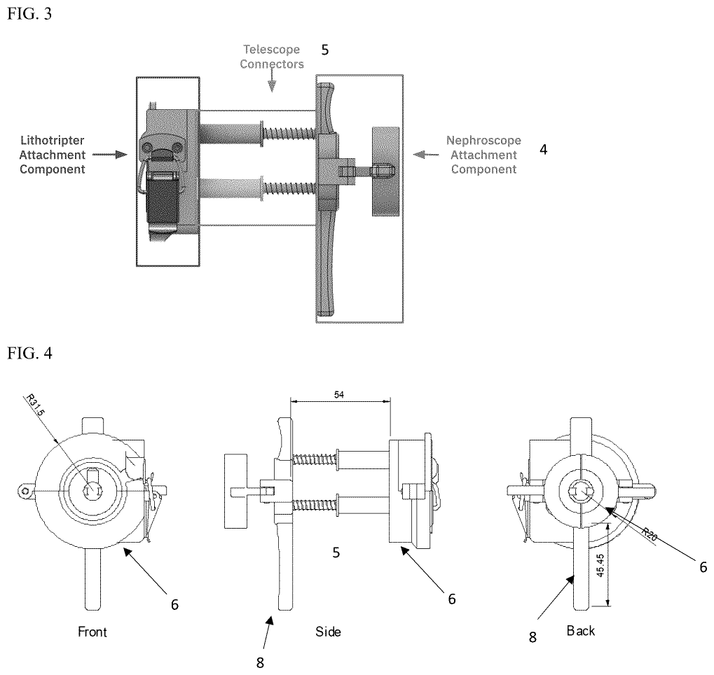

Details of the exemplary, non-limiting, devices shown in FIGS. 3-16 are described below. FIG. 3 shows an overview of an exemplary assembly comprising a lithotripter attachment device 6, a nephroscope attachment device 4, and a plurality of connectors 5 extending between the lithotripter attachment device 6 and the nephroscope attachment device 4. In the illustrated embodiment, the connectors 5 are telescopic. As shown in FIG. 3, the telescope connector 5 joins the lithotripter attachment device 6 and the nephroscope attachment device 4. The lithotripter attachment device 6 is positioned in the rear of the assembly. The lithotripter attachment device 6 is designed to lock onto the lithotripter during use and hold it in place with no rotation.

FIG. 4 shows an overview of exemplary assembly with dimensions. The present disclosure is not limited to particular dimensions. Dimensions (in mm) shown in figures described herein are non-limiting examples of suitable dimensions. Still referring to FIG. 4, shown is a front view showing lithotripter attachment device 6 and nephroscope attachment device 4, a side view showing lithotripter attachment device 6, telescope connectors 5, and nephroscope attachment device 4, and a back view showing lithotripter attachment device 6 and nephroscope attachment device 4.

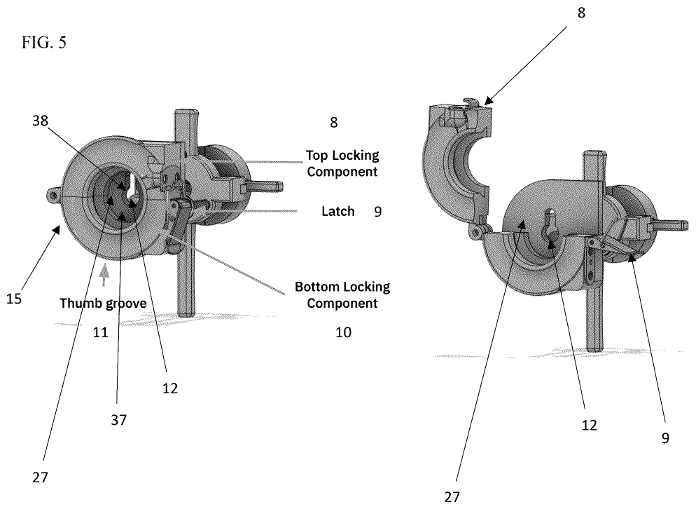

Now referring to FIG. 5, shown are details of the locking mechanism of lithotripter attachment device 6. A locking assembly 15 comprises a first collar portion 8, a second collar portion 10, a releasable latch 9, and a thumb groove 11. The left panel of FIG. 5 shows the locking assembly 15 in a closed position with first collar portion 8 and the second collar portion 10 held together by the releasable latch 9 in a closed position. In the closed position, the first collar portion 8 and the second collar portion 10 form a closed collar (e.g., a 360 degree collar). The right panel of FIG. 5 shows the locking assembly 15 in an open position. The releasable latch 9 is in an open position, allowing first collar portion 8 to be released and rotated away from second collar portion 10.

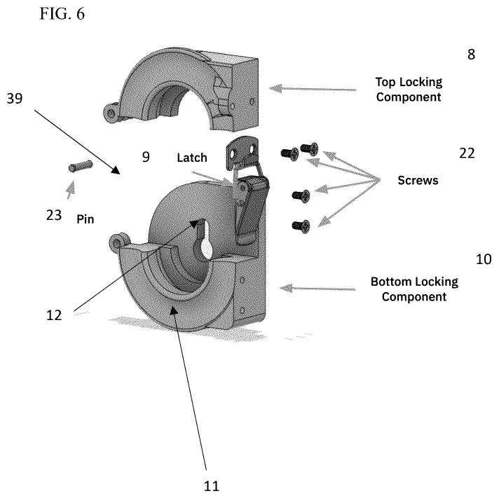

Still referring to FIG. 5, in use, a lithotripter (not shown) is inserted by undoing the latch 9 positioned on the side of lithotripter attachment device 6, lifting the first collar portion 8 into the open position (right image of FIG. 5), and placing the lithotripter into an opening 27. A keyhole shaped opening 12 is positioned on a wall (37) extending from the second collar portion 10. The keyhole shaped opening 12 allows the front lip of a lithotripter to be able to go over the bottom of lithotripter attachment device 6 of and slide down into the contoured groove 38 once it is fully in. Once the lithotripter is in position on the second collar portion 10, the top collar portion 8 is then clamped down and latched back into place using latch 9. In some embodiments, on the back of lithotripter attachment device 6 there is a thumb or finger groove 11 (FIG. 6) located on the first and second collar portions that extends at least partially circumferentially around the lithotripter. In the illustrated embodiment, the groove 11 extends entirely circumferentially around the lithotripter. When in use, this is the place where the user's thumb or finger is placed. The thumb can be placed either above, below, or on the sides of the groove depending on preference. The groove continues all the way around the rear in order to be able to accommodate both right- and left-handed users.

Now referring to FIG. 6, shown is an expanded view of locking assembly 15 of lithotripter attachment device 6. Shown is first collar 8, second collar 10, releasable latch 9, and thumb groove 11. Also shown are latch screws 22 that secure the latch 9 to the first collar 8 and second collar 10. Further shown is locking pin 23 that seals locking mechanism 15 in the closed position. Pin 23 defines a pivot axis 39 about which the first collar portion 8 moves with respect to the second collar portion 10.

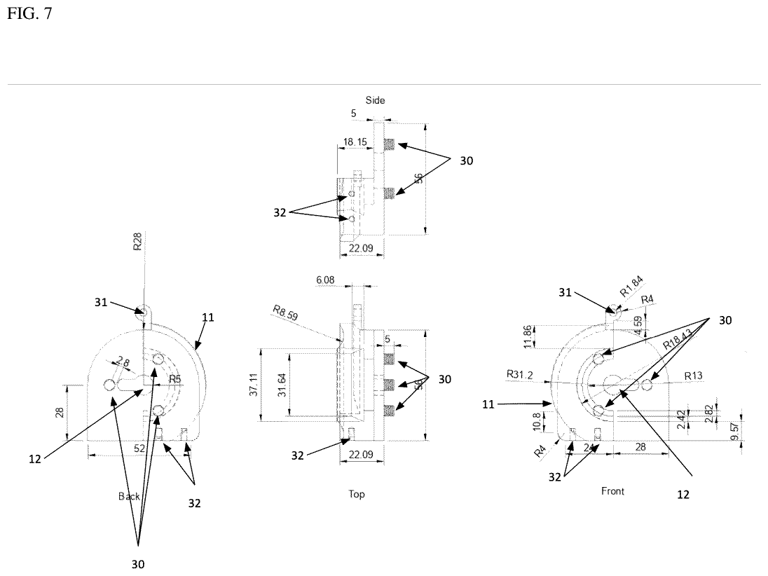

Now referring to FIG. 7, shown is a labeled drawing of second collar 10 of lithotripter attachment device 6 with dimensions in mm. The left panel is a back view showing keyhole opening 12, inner telescope arm attachment holes 30, and pin hole 31. The middle panel shows top view with latch screw attachment holes 32 and side view of latch 9 and outer telescope arm attachment holes 30. The right panel shows a front view with latch screw attachment holes 32, outer telescope arm attachment holes 30, and pin hole 31.

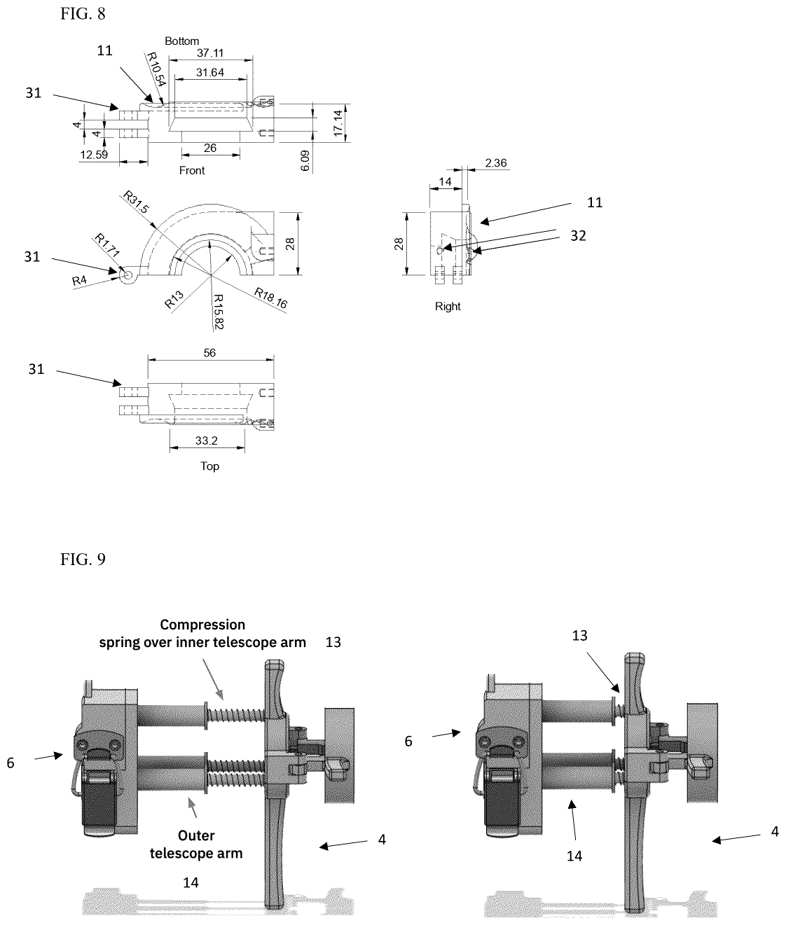

Now referring to FIG. 8, shown is a labeled drawing of first collar 8 of lithotripter attachment device 6 with dimensions in mm. Shown are front and top edges of first collar 8 with dimensions, side view with pin hole 31, and right side view with latch screw attachment holes 32.

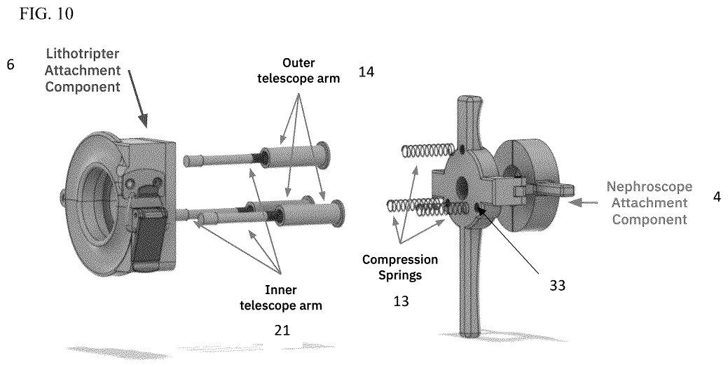

Now referring to FIG. 9, the assembly is shown in a neutral configuration (left) and a compressed configuration (right panel). The telescoping connectors 5 connects the lithotripter attachment device 6 and the nephroscope attachment device 4. The telescoping connector 5 comprises a plurality of outer telescope arms 14, a plurality of inner telescope arms 21 (See FIG. 10), and a plurality of compression springs 13. In its resting position (left panel of FIG. 9), the outer telescope arms 14, inner telescope arms 21, and biasing members (e.g., compression springs) 13 around them are fully extended with minimal compression on the springs. Once pressure is placed on the thumb groove of the lithotripter attachment device, the compression springs 13 compress and the inner telescoping arms 21 moves within the outer telescoping arms 14, protruding the lithotripter from within the nephroscope working channel (right panel of FIG. 9). Once the pressure is taken off, the springs push the device back into the resting position (left panel of FIG. 9).

The present disclosure is not limited to a particular number of outer telescope arms 14, inner telescope arms 21, and compression springs 13. In FIG. 9, a device with 3 sets of outer telescope arms 14, inner telescope arms 21, and compression springs 13 is shown. However, other numbers of sets of outer telescope arms 14, inner telescope arms 21, and compression springs 13 are specifically contemplated (e.g., 1 or more, 2 or more, 3 or more, 4 or more, 5 or more, etc.).

Now referring to FIG. 10, shown is a detailed view of disassembled telescoping connector 5. Shown are outer telescope arms 14, inner telescope arms 21, and compression springs 13. Also shown are compression spring attachment holes 33. Compression springs 13 are inserted into compression spring attachment holes 33 in order to attach nephroscope attachment device 4 to telescoping connector 5.

FIG. 11 shows an additional view of disassembled telescoping connector 5, nephroscope attachment device 4, and lithotripter attachment device 6. Shown are outer telescope arms 14, inner telescope arms 21, and compression springs 13. Also shown are compression spring attachment holes 33. Still referring to FIG. 11, shown are details of lithotripter attachment device 6, including latch 9, pin 23, keyhole opening 12, and pin holes 31. Still referring to FIG. 11, shown are details of nephroscope attachment device 4 (see below for a description of nephroscope attachment device). Shown are compression spring attachment holes 33, top or index finger handle 16, bottom handle 18, and nephroscope locking assembly 28.

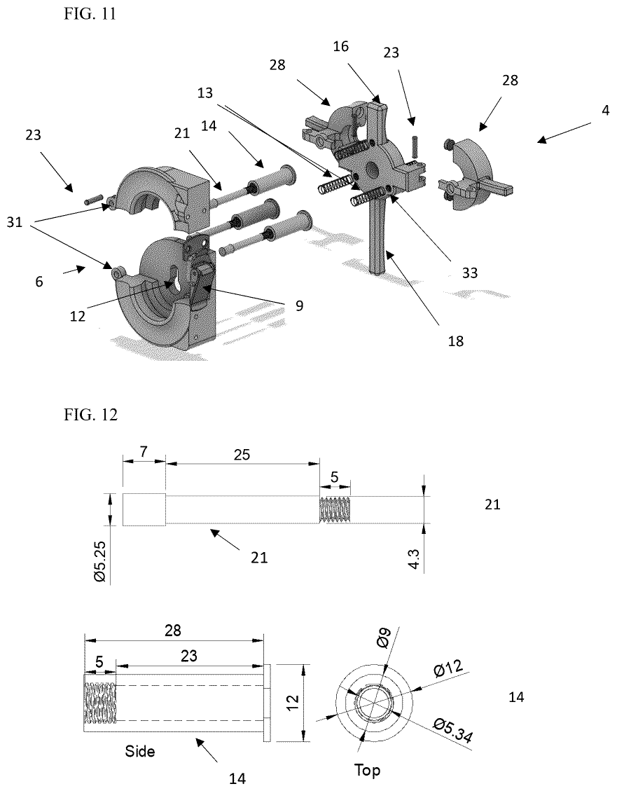

Now referring to FIG. 12, shown is a labeled drawing of inner telescoping arm 21 (top) and outer telescoping arm 14 (bottom) with dimensions in mm.

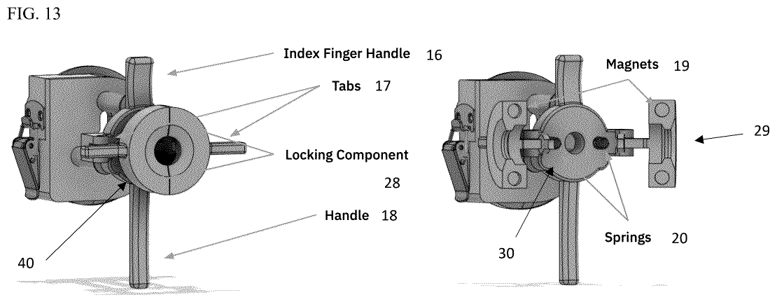

Now referring to FIG. 13, shown is nephroscope attachment device 4. The nephroscope attachment device 4 comprises base 40 at least one handle (e.g., shown in FIG. 13 as top or index finger handle 16 and bottom handle 18), nephroscope locking assembly 28, magnets 19, and springs 20. The nephroscope attachment device 4 provides secure attachment of the device to a nephroscope. The nephroscope locking assembly 28 comprises a set of tabs 17, along with magnets 19 and biasing member (e.g., springs) 20. In some embodiments, the nephroscope attachment device 4 comprises a first tab 17 pivotably connected to the base 40 about a first axis and a second tab 17 pivotably connected to the base about a second axis. In some embodiments, the second axis is spaced apart from and parallel to the first axis.

Without the nephroscope attached (right panel of FIG. 13), nephroscope locking assembly 28 is in an open configuration. The front plate 29 is held open from back plate 30 with springs 20 located in the back plate of the nephroscope locking assembly 28.

Still referring to FIG. 13, When the nephroscope is placed within the device, it is moved down the probe of the already inserted lithotripter, and into the opened nephroscope locking assembly 28. The cap on the nephroscope catches the nephroscope locking assembly 28 and rotates the front and back plates closer to each other, locking it in place after the magnets connect. The top handle 16 is used to hold the index finger on the top and any number of the remaining fingers on the bottom handle 18. The tabs are in place so that when the user needs to remove the nephroscope from the device, they are able to pull back on either of the tabs, release the magnets from each other and pull the nephroscope out.

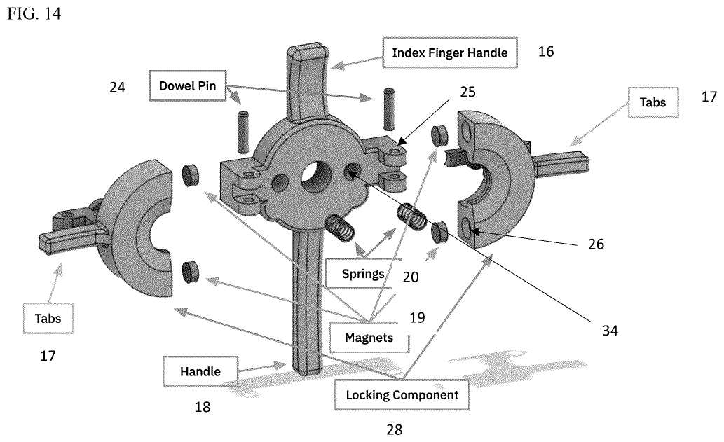

Now referring to FIG. 14, shown is a detailed view of disassembled nephroscope attachment device 4. Shows is top or index finger handle 16, bottom handle 18, nephroscope locking assembly 28, magnets 19, springs 20, tabs 17, and dowel pin 24. Also shown is dowel pin hole 25, spring hole 34, and magnet attachment groove 26. The magnet attachment groove 26 secures magnets 19 in a proper location for securing locking assembly 28. The dowel pin 24 slides into dowel pin hole 25 to secure locking assembly 28 to the base of the device.

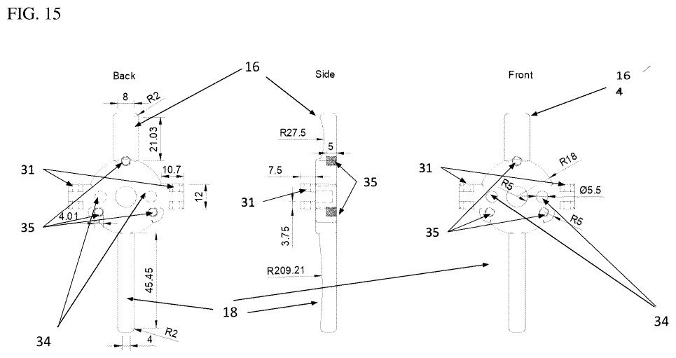

Now referring to FIG. 15, shown is a labeled drawing of nephroscope locking assembly 28 with dimensions in mm. Shown are back, side, and front views showing top or index finger handle 16, bottom handle 18, spring hole 34, and inner telescope arm attachment holes 35.

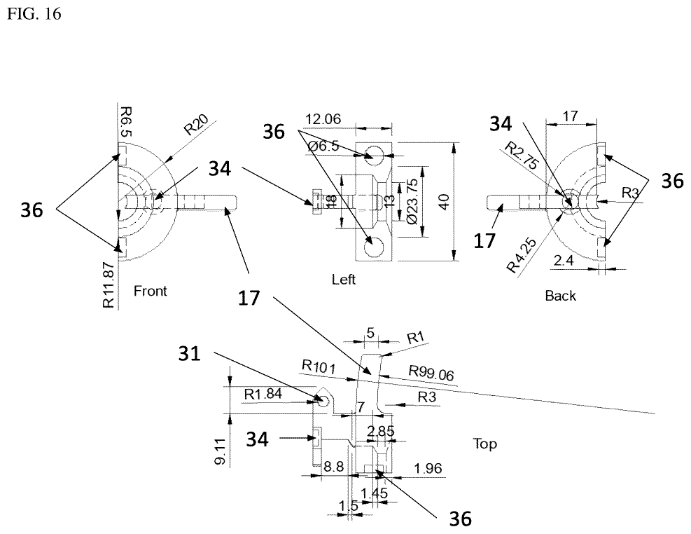

Now referring to FIG. 16, shown is a labeled drawing showing details of locking assembly 28 of nephroscope attachment device 4 with dimensions in mm. Shown are tabs 17, pin hole 31, spring hole 34, and magnet holes 36.

Devices are constructed of any suitable material. In some embodiments, the device is 3D printed using polylactic acid (PLA) or liquid photopolymer resin, specifically Tough 1500 and 200 resin to provide extra strength to our device. The compression springs (stainless steel), pins (alloy steel), draw latches (stainless steel), and screws (metric brass) are obtained from available suppliers.

All publications and patents mentioned in the above specification are herein incorporated by reference in their entirety for all purposes. Various modifications and variations of the described compositions, devices, systems, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

1. An assembly comprising:
a) a lithotripter attachment device comprising a lithotripter locking assembly;
b) a nephroscope attachment device comprising a nephroscope locking assembly; and
c) a telescoping connector connecting said lithotripter attachment device and said nephroscope attachment device.

2. The assembly of claim 1, wherein said lithotripter locking assembly comprising a first collar portion, a second collar portion, and a releasable latch, wherein said latch holds said first collar portion and said second collar portion together when said latch is in a closed configuration.

3. The assembly of claim 1, wherein said lithotripter attachment device further comprises one or more additional components selected from the group consisting of a thumb groove and a keyhole shaped opening.

4. The assembly of claim 1, wherein said telescoping connector comprises an outer telescope arm, an inner telescope arm, and a biasing member.

5. The assembly of claim 1, wherein the telescoping connector is one of a plurality of telescoping connectors connecting the lithotripter attachment device and the nephroscope attachment device.

5. The assembly of claim 4, wherein said plurality of telescoping connectors includes three telescoping connectors.

6. The assembly of claim 1, wherein said nephroscope attachment device further comprises one or more components selected from the group consisting of an top handle and a bottom handle.

7. The assembly of claim 1, wherein said nephroscope locking device comprises a set of a front pate, a back plate, tabs, magnets, and springs.

8. The assembly of claim 1, wherein the nephroscope locking device includes a base with at least one handle, a first tab pivotably connected to the base about a first axis, a second tab pivotably connected to the base about a second axis.

9. The assembly of claim 8, wherein the second axis is spaced apart from and parallel to the first axis.

10. The assembly of claim 8, wherein the first tab includes at least one permanent magnet.

11. The assembly of claim 8, further comprising a bias member positioned between the base and the first tab.

12. A method of performing percutaneous nephrolithotomy, comprising:
a) securing a lithotripter and a nephroscope in an assembly of claim 1; and b) performing percutaneous nephrolithotomy with the lithotripter and a nephroscope.

13. The method of claim 12, wherein said assembly provides control of the forward and backward movements of the lithotripter within the working channel of the nephroscope.

14. The method of claim 12, wherein the assembly device can be controlled with only hand and wrist movement.
